(19) United States
(12) Patent Application Publication
(10) Pub. No.: US 2017/0199197 A1
(43) Pub. Date: Jul. 13, 2017

(54) DIAGNOSIS OF CANCEROUS CONDITIONS

(71) Applicant: ABA GMBH & CO. KG, Dortmund (DE)

(72) Inventor: Sylvain TOUREL, Hattingen (DE)

(73) Assignee: ABA GMBH & CO. KG, Dortmund (DE)

(21) Appl. No.: 15/314,523

(22) PCT Filed: May 29, 2015
(86) PCT No.: PCT/EP2015/062050

§ 371 Date: Nov. 29, 2016

(30) Foreign Application Priority Data
May 29, 2014 (EP) 14170472.6

(51) Int. Cl. G01N 33/574; B01L 3/00

(52) U.S. Cl. CPC G01N 33/57492; B01L 3/502761; B01L 2300/0654

(57) ABSTRACT

The invention relates to a method for diagnosing cancer and use thereof, in particular for providing profiles and use thereof.

The invention relates to a method for diagnosing cancer and use thereof, in particular for providing profiles and use thereof.

Cancer is a class of diseases characterised by uncontrolled cell growth and the spread of abnormal cells in the body, and in the case of metastasis eventually leads to death of the patient.

The term “cancer and cancerous conditions” summarises a class of diseases which have the common factor that they form malignant tumours. To date, more than 200 different tumours have been identified. What is characteristic for all malignant tumours is the uncontrolled proliferation of cells, the ability to displace healthy tissue (invasion of the adjacent tissue), and the ability to form metastases in the tissue of the entire body (distant metastases). These three processes are characteristic for the progression of cancer and are used as criteria for classifying cancer into cancer stages I, II, III and IV (or A-D) and for staging by TNM classification and determining the aggressiveness of the disease. From stage III (C), it is possible to determine that the tumour is outgrowing its area of original formation and is displacing the surrounding tissue. From stage IV (D), distant metastases can be determined. Depending on the stage of cancer, different treatment is recommended so as to achieve a successful result. Early identification and assessment of the aggressiveness of the tumour cells are therefore of great importance in order to select a suitable course of therapy for a patient. Cancerous conditions are diagnosed in particular by imaging methods, such as MRI, CT and the like, and by the detection of specific tumour markers. Tumour markers are predominantly proteins or peptides which are detected in the blood or other bodily fluids of the patient or on the cell surface and of which an increased concentration indicates a tumour.

Since malignant tumour cells develop from mutated cells of normal tissue, the tumour markers can also be detected on cells of normal tissue and are characterised in tumours merely by their different frequency. A large number of different tumour markers have been associated with various cancerous conditions.

“CTCs” have proven to be particularly suitable cancer cells in the early diagnosis. The cells are, in particular, circulating tumour cells (CTCs for short), mesenchymal stem cells from peripheral blood or other bodily fluids, and also disseminated tumour cells from bone marrow (DTCs for short) (referred to jointly hereinafter as “CTCs”).

CTCs are slightly larger than the blood cells in blood, for example red or white blood cells or also blood platelets, and can be enriched by means of filters (for example by means of parylene) from patient blood and test subject blood (DE 20 2012 003 212 WO2010/135603), even if there is only one CTC in 10E10 blood cells in peripheral blood, as can be the case in cancer patients. DE 10 2010 032 081 A1 describes a membrane filtration process for enrichment and isolation of CTCs for the purpose of tumour diagnosis.

The occurrence of CTCs in peripheral blood is an indication of a possible scattering of cells of a solid tumour at a very early stage, at which it is still not possible to detect any metastasis using conventional imaging examination methods (CT, etc.). Both the detection and characterisation of CTCs in peripheral blood are therefore very promising approaches for identifying systemic tumour cell propagation very early on and for using CTCs as prognostic markers. Prognoses could thus be made and continuous monitoring of systemic therapies carried out. Furthermore, the characterisation and assessment of CTCs as a diagnostic instrument could also be used to select a suitable treatment for solid tumours.

Proceeding from this prior art, the inventors set themselves the task of providing a new method for diagnosing cancer cells, in particular CTCs.

Surprisingly, a diagnosis method can be carried out by means of the determination of retention times of cancer cells.

In the sense of this invention, the terms tumour, cancer or cancer cells, and tumour cells are to be understood synonymously. However, CTCs are preferred in accordance with the invention.

“Retention times” in the sense of this invention means the time required by the cancer cells to pass through a suitable “column” (from injection to detection). In accordance with the invention, “retention” means the delayed throughflow of individual cancer cells in the mobile phase by interaction with the stationary phase, as is usual in chromatography. Cancer cells which have no interaction with a stationary phase correspond to the dead time of the mobile phase. From the retention times, it is possible to determine the speed. The mean retention time from several runs can also be determined as the mean speed of the cancer cells.

The mobile phase can be any carrier having a throughflow rate, such as gas or liquid, which can have a normal gradient. Buffers, such as PBS inter alia, and fluids which are suitable for cancer cells, such as growth media, for example DMEM growth medium, RPMI growth medium, inter alit, are particularly suitable as mobile phase buffers. The gradient can be arbitrary, in particular a concentration gradient or pressure gradient. Suitable pumps which are particularly suitable for microfluidics (below) are commercially available for example from the company Fluigent.

A “column” in the sense of this invention is preferably a “microfluidic” device (also referred to as a “chip”), in particular containing a micro channel or micro capillary. Such micro channels very particularly preferably have the diameter of a cancer cell (approximately 10 to 30 μm). The length of such a micro channel can vary from 1 mm to 10 cm or more. The width is for example 10 to 150 μm or also more to 1,000 μm. Such micro channels are usually rectangular in cross-section (for example 80×20 μm). Channels which have a depth of approximately 20 μm, in particular 10 to 30 μm, are preferred in accordance with the invention.

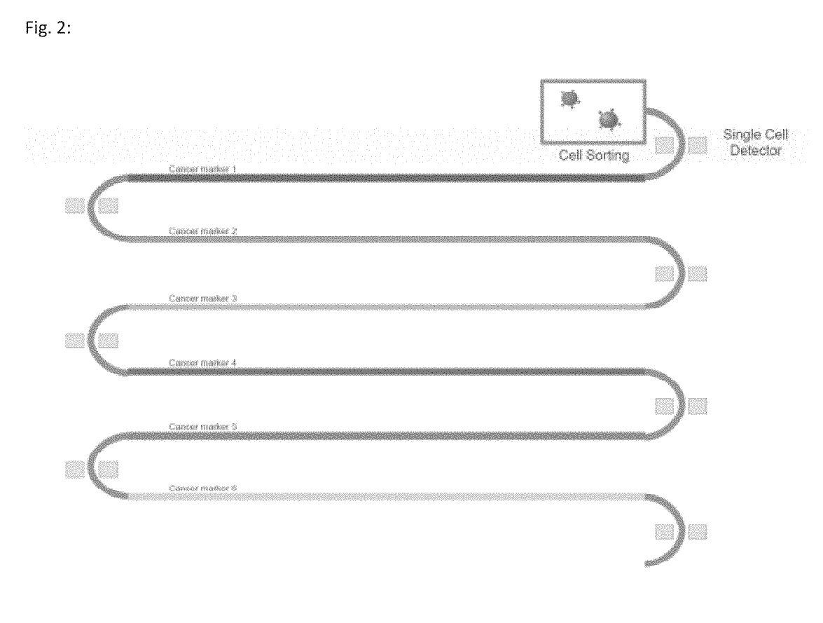

The throughput rate is preferably 0.02 μl/min to 10 μl/min. The microfluidic device also contains a suitable sample receptacle. A suitable microfluidic device can be purchased for example, more specifically in the form of what are known as “straight channel chips” made of PMMA or Topas from Microfluidic ChipShop. The microfluidic device preferably consists of a transparent material, and the design, i.e. the routing of the micro channel, can be arbitrary, but a channel routing according to FIG. 2 is preferred, such that each straight portion can be easily provided with a different stationary phase.

Any produced microfluidic device can be provided with a barcode.

The cancer cells entering the column and/or the cancer cells exiting from the column can be detected (=cell detection) using conventional means known to a person skilled in the art (light barriers, electrodes, conductivity measurement), such that the retention times and the speeds can be determined reliably and precisely. Furthermore, the cancer cells for detection can be labelled using a sensor, for example a fluorescence labelling, a fluorophore (for example BISBENZIMIDE HOECHST NO 33342 TRIHYDROCL 1), or can have another label.

The cell detection can also be performed optically on the basis of microfluidics, for example using a microscope or a fluorescence microscope. Optical detection is possible on the basis of contrast differences.

In a particularly preferred embodiment, the cell detection is performed by means of computer-assisted readout, more specifically a microscope combined with a camera (what is known as a microscope-camera, for example inverted microscope Leica (objective lenses 4 and 10), inter alia) with use of cell detection software (for example Medealab, Erlangen, see the example).

Such cell detection software allows the computer-assisted detection of cancer cells in an arbitrary window (section) in the micro channel, so that the cancer cells can be selected individually and a movement analysis can be performed. The (mean) retention time and (mean) speed of the cancer cells can also be determined on the basis of such a window.

Said cancer cells have specific tumour markers or cell surface molecules on their surface, such as CD45, Pan-CK, CD133, N-cadherin, E-cadherin, aSMA, ASGPR1, Twist, C-Met, epithelial cell adhesion molecule (EpCAM, CD326), carcioembryonic antigen (CEA), alpha ferroprotein (AFP), carbohydrate antigen 19/9 (CA 19-9), cancer antigen 72/4 (CA 72-4), cancer antigen 125, cancer antigen 15/3 (CA 15-3), neuron-specific enolase (NSE), squamous cell carcinoma antigen (SCC), cytokeratin fragment (CYFRA), human chorionic gonadotropin (HCG), prostate-specific antigen (PSA), human thyroglobulin (HTG), and mucin-like cancer associated antigen (MCA). Further tumour markers can be deduced from the general literature. Due to these tumour markers, dynamic interactions can occur with the stationary phase. However, in accordance with the invention, particularly preferred stationary phases are those that particularly preferably comprise the receptors for tumour markers, such as antibodies (polyclonal or monoclonal), aptamers, peptides, proteins or other receptors for tumour markers known to a person skilled in the art.

The greater the interactions between the cancer cells in the mobile phase and the stationary phase, the longer is the characteristic retention time. The required time is advantageously directly proportional to the amount of tumour markers contained on a cancer cell and allows the specific profiling or the creation of a profile (fingerprint) of at least one examined cancer cell, in particular CTC.

It is also preferred that the stationary phase has different receptors for tumour markers over a number of areas; see FIG. 2 by way of example. These areas can preferably be designed in succession on the stationary phase.

The invention therefore relates to a method for diagnosing cancer or characterising cancer cells, in which the retention times of cancer cells, preferably CTCs, are determined, wherein the retention time is determined by means of a microfluidic device, in particular by means of a micro channel (also micro capillary) and has at least one receptor for a tumour marker, in particular a number of areas of different receptors for tumour markers. In order to produce such areas, linkers known to a person skilled in the art can be used, such as biotin/(strept)avidin, and the like, such that the receptor is sufficiently fixed in the stationary phase. For example, a biotin-aptamer/antibody (receptor) can thus be fixed/coupled with provided streptavidin (see the example). Of course, other coupling systems are known appropriately to a person skilled in the art, such as molecular imprinting (Seung-Woo Lee, Toyoki Kunitake, Handbook of Molecular Imprinting: Advanced Sensor Applications, CRC-Press (2012)).

In this way, a microfluidic device can be coated by one or more of the same or different receptor(s) for tumour markers. In other words, the micro channel is formed accordingly with a modified stationary phase, or “coating” above and hereinafter. Aptamers are preferably used, since the correct conformation can be reproduced or produced in situ by means of refolding (for example 5 min. at 85 degrees C. with subsequent cooling to room temperature).

In a further preferred embodiment the microfluidic device can be formed in such a way that areas of the micro channel have coatings and other areas do not have a coating.

Once microfluidic device can also have a coating and a further microfluidic device of the same design can have no coating.

Within the scope of the preferred method for cell detection by means of computer-assisted readout by means of a microscope-camera, a first window can have no coating and a second or further window can have a coating.

This procedure allows comparative tests or serves as a control/reference.

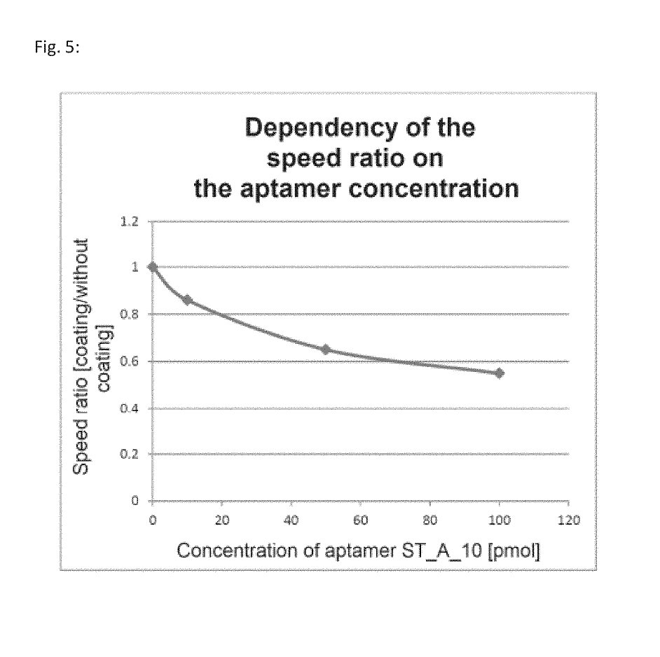

Depending on the used microfluidic device, a calibration can be performed for example depending on the coating. FIG. 5 shows, in an exemplary manner, a calibration curve and allows the calculation of the equilibrium constant or the like. Consequently, the method according to the invention can be standardised and allows the repeatable reproduction of the measurement results ((mean) retention times or (mean) speeds of the cancer cells).

The results can be detected arbitrarily depending on the test parameters (microfluidics, coating, cancer cell, tumour marker, etc.) and can be systematised and presented in profiles. Such profiles can be assigned to one or more patients/test subjects, including within the scope of personalised medicine or with existing data (for example “big data” or “data clouds”), and for example can be compared to another tumour diagnosis.

The individual profiles from the obtained measurement results ((mean) retention times or (mean) speeds of the cancer cells) can be collated and stored in a (electronic) database. By way of example, the various profiles can be compared with one another on the basis of the database and can be assessed systematically.

This advantageously allows a risk stratification or therapy management, since for example metastases and other risks can be identified. Furthermore, patients can advantageously be stratified according to risk groups.

In a further embodiment the invention therefore relates to a method for risk stratification of test subjects/patients. The term “risk stratification” in accordance with the invention comprises the detection of patients, in particular emergency patients and risk patients, who have a poorer prognosis for the purpose of more intensive diagnosis and therapy/treatment of cancerous conditions with the objective of enabling the most favourable progression possible of the disease. A risk stratification according to the invention consequently allows an effective treatment process of cancerous conditions by means of cancer drugs.

The invention therefore also relates to the identification of patients with increased risk and/or an unfavourable prognosis of cancerous conditions, more specifically in the case of symptomatic and/or asymptomatic patients, in particular emergency patients.

In particular in cases of emergency and/or intensive care, reliable stratification particularly advantageously can be provided by means of the method according to the invention. The method according to the invention thus enables clinical decisions which lead to rapid therapy success and to the avoidance of instances of death. Such clinical decisions also comprise further treatment by means of drugs for the treatment or therapy of cancerous conditions.

The invention therefore also relates to a method for the diagnosis and/or risk stratification of patients of cancerous conditions in order to make clinical decisions, such as further treatment and therapy by means of drugs, in particular in time-critical intensive care or emergency care, including the decision to hospitalise the patient.

In a further preferred embodiment the method according to the invention therefore relates to the therapy management of cancerous conditions.

In a further preferred embodiment the invention relates to the diagnosis and/or risk stratification for the prognosis, differential diagnostic early detection and detection, assessment of the degree of severity, and assessment alongside therapy of the progression of a cancerous condition, wherein, by means of the profiling performed in accordance with the invention, the diagnosis, prognosis, stratification and/or detection can be carried out quickly and reliably, where appropriate with use of a database.

The invention also relates to the use of a kit comprising a microfluidic device according to the invention for carrying out the method according to any one of the above embodiments, where appropriate together with further conventional aids.

The invention also relates to a kit comprising a microfluidic device according to the invention for carrying out the method, together with means for computer-assisted readout of the microfluidic device, in particular a microscope combined with a camera (what is known as a microscope-camera) and cell detection software together with a conventional computer (IT hardware) and database.

With regard to general literature relating to the microfluidics for cancer cells for the purpose specified, reference can be made to the following documents: Microfluidic approaches for cancer cell detection, characterization, and separation, Lab Chip. 2012 Apr. 24; 12(10):1753-67, Chen J, Li J, Sun Y; Aptamer-based microfluidic device for enrichment, sorting, and detection of multiple cancer cells, Anal Chem. 2009 Sep. 1; 81(17):7436-42, Mu Y, Phillips J A, Yan J, Li Q, Fan Z H, Tan W; Capturing cancer cells using aptamer-immobilized square capillary channels, Mol Biosyst. 2011 May; 7(5):1720-7, Martin J A, Phillips J A, Parekh P, Sefah K, Tan W; Highly efficient circulating tumor cell isolation from whole blood and label-free enumeration using polymer-based microfluidics with an integrated conductivity sensor, J Am Chem Soc. 2008 Jul. 9; 130(27):8633-41, Adams A A, Okagbare P I, Feng J, Hupert M L, Patterson D, Göttert J, McCarley R L, Nikitopoulos D, Murphy M C, Soper S A; Probing circulating tumor cells in microfluidics, Lab Chip. 2013 Feb. 21; 13(4):602-9, Li P, Stratton Z S, Dao M, Ritz, J, Huang T J; High-throughput selection, enumeration, electrokinetic manipulation, and molecular profiling of low-abundance circulating tumor cells using a microfluidic system, Anal Chem. 2011 Mar. 15; 83(6):2301-9, Dharmasiri U1, Njoroge S K, Witch M A, Adebiyi M G, Kamande J W, Rupert M L, Barany F, Soper S A; Continuous separation of cells and particles in microfluidic systems, Chem Soc Rev. 2010 March; 39(3):1203-17, Lenshof A, Laurell T.

The invention also relates to a method in which, in a first step, cancer cells are enriched from a body sample, and only then are the retention times determined in accordance with the invention. This can be implemented for example with a cell sorter or separator. However, DEP (di-electrophoresis enrichment/sorting of CTC is particularly preferred: An integrated dielectrophoretic chip for continuous bioparticle filtering, focusing, sorting, trapping, and detecting, Biomicrofluidics, 2007 May 10; 1(2):21503, Cheng I F, Chang H C, Hou D, Chang H C; Microelectromechanical Systems, journal of (Volume: 16, Issue: 4), A Programmable Biochip for the Applications of Trapping and Adaptive Multisorting Using Dielectrophoresis Array, Ting-Chen Shih; Nat. Tsing Hua Univ., Hsinchu, Kuang-han Chu; Cheng-Hs en Liu; A microfabrication-based dynamic array cytometer, Anal Chem. 2002 Aug. 15; 74(16):3984-90, Voldman J, Gray M L, Toner M, Schmidt M A; Label-free cell separation and sorting in microfluidic systems, Anal Bioanal Chem. 2010 August; 397(8):3249-67, Gossett D R, Weaver W M, Mach A J, Hur S C, Ise H T, Lee W, Amini H, Di Carlo D; Cell Separation by Non-inertial Force Fields in Microfluidic Systems; Mech Res Commun. 2009 Jan. 1, 36(1):92-103, Tsutsui H, Ho C M; A scalable addressable positive-dielectrophoretic cell-sorting array, Anal Chem. 2005 December. 15; 77(24):7976-83, Taff B M, Voldman J; A CMOS chip for individual cell manipulation and detection, Solid-State Circuits, IEEE Journal of Volume: 38, Issue: 12), Manaresi, N., Silicon Biosystems s.r.l, Bologna, Italy, Romani, A. Medoro, G., Altomare, L.

The sample can be prepared from bodily fluids, in particular blood, serum, plasma, or urine of a test subject/patient, as described for example for CTC in EP 2 706 357 or above. The method according to the invention is performed ex vivo/in vitro.

### EXAMPLES AND FIGURES

These examples serve exclusively to explain the invention and do not limit the invention to these examples.

### EXAMPLES

### Example 1

Mikrofluidics (“chip”)

Straight Channel Chips made of PMMA from Microfluidic ChipShop, Interface Olive, silicone tube ID=approx. 1 mm, OD=approx. 0.75 mm. The silicone tube is completely filled with sterile-filtered PBS. One end is connected to the chip via the olive, the other end is inserted into a 50 ml tube containing approx. 25 ml PBS and is secured. The cells are controlled by the force of gravity by lifting and lowering the 50 ml tubes (an exact control of the cell is thus possible). The 50 ml tubes are placed on stand clamps in order to achieve the same flow speed.

### Example 2

Production of Profiles:

Jurkat cells were cultured in RPMI medium (5% CO₂, 37 degrees C.) (RPMI Medium with penicillin, streptomycin and L-glutamine and 25 mM Hepes (order number Lonza: 09-774E), 10 ml 100 mM Na-Pyruvat (order no. Lonza BE13-115E), and 50 ml BBS (order number VWR: BCHRS0615)).

Capillaries from the company ChipShop were used and coated with polystreptavidin R coating kit (Biotez, Berlin, product no: BTCK-MC0020) according to the instructions. This coating is produced in 5 steps: pre-coating, 8 degrees C., incubated overnight, washed 5 times with 0.9% NaCl solution, polystreptavidin R 50 ug/mL, 8 degrees C., incubated overnight, washed 5 times with dd water, and the coating was dried for 2 h at room temperature.

Fixing the aptamer SGC8 (5′-Cy5, 3′-biotin) (aptamers evolved from live cells as effective molecular probes for cancer study, Proc Natl Acad Sci USA. 2006 Aug. 8; 103(32):11838-43. Epub 2006 Jul. 27, Shang guar D1, Li Y, Tang Z, Can Z C, Chen H W, Mallikaratchy P, Sefah K, Yang C J, Tan K) to the capillary wall by biotin/streptavidin binding.

SSCP is a specific aptamer with respect to Jurkat CEM cells with folding at 95 degrees C., 5 min end 95 C→20 degrees C., 2 degrees C./min. Steps:

The refolded aptamers were fixed by means of 1 ml syringe, more specifically to half the capillary length, 10 min incubation at 20 degrees C., washing 5 times with PBS, centrifuge 1 Jurkat cell cultures at 500 g for 5 min, remove supernatant, wash 2 times with 1 mL PBS, cell concentration is determined with microscope, 100 cells are injected into capillary connection by means of insulin syringe, capillary connection is connected to a syringe pump with a throughput of 0.5 μl/min., speed of the individual cells is measured by means of inverted microscopy (timer), wherein portions with coating slow the cells in accordance with the invention, as demonstrated below:

Throughput rate 3 μl/min

| without | | | |
|---|---|---|---|
| coating | retention | distance | speed |
| measurement | time [s] | [mm] | [μm/s] |
| 1. | 80 | 10 | 125 |
| 2. | 76 | 10 | 132 |
| 3. | 68 | 10 | 147 |
| 4. | 65 | 10 | 154 |
| 5. | 62 | 10 | 161 |
| Average | 70.2 | 10 | 142 |
| with | | | |
| coating | Retention | Distance | Speed |
| Measurement | time [s] | [mm] | [μm/s] |
| 1. | 77 | 10 | 130 |
| 2. | 80 | 10 | 125 |
| 3. | 79 | 10 | 127 |
| 4. | 80 | 10 | 125 |
| 5. | 90 | 10 | 111 |
| 6. | 77 | 10 | 130 |
| Average | 80.5 | 10 | 124 |

Throughput rate 2 μl/min

| | Retention | Distance | Speed |
|---|---|---|---|
| | time [s] | [mm] | [μm/s] |
| without | | | |
| coating | | | |
| Measurement | | | |
| 1. | 110 | 10 | 90,909 |
| 2. | 103 | 10 | 97,087 |
| 3. | 100 | 10 | 100,000 |
| 4. | 97 | 10 | 103,093 |
| 5. | 94 | 10 | 106,383 |
| Average | 100.8 | 10 | 99,206 |
| with | | | |
| coating | | | |
| Measurement | | | |
| 1. | 126 | 10 | 79,365 |
| 2. | 126 | 10 | 79,365 |
| 3. | 124 | 10 | 80,645 |
| 4. | 123 | 10 | 81,301 |
| 5. | 115 | 10 | 86,957 |
| Average | 122.8 | 10 | 81,433 |

The presented results represent a profile or fingerprint according to the invention.

### Example 3

Description of cell detection by means of computer-assisted readout, more specifically a microscope in combination with a camera (microscope-camera), with use of cell detection software.

The use of cell detection software media AV Multimedia and Software GmbH, Erlangen, Germany (medeatAB Tracking) allows the specific movement analysis and the counting of cells and assessment thereof.

Camera: Basler acA2040-90 um USB 3.0-Kamera with the CMOSIS CMV4000 CMOS sensor
90 frames per second (fps), 4 MP (megapixels), monochromatic resolution of 2048×2048 pixels (px) (pixel size: 5.5 μm×5.5 μm)
Adjustments with transmitted light: Gain: 0, Gamma: 1.0, Exposure Time: 42.0, frame rate: 30 fps
Tracking options: match factor: 2.5, runtime: adaptable in a flexible manner, image raster: 1, real time [on/off]:
Search positions: 10.000

For example, measurement of an 800×20 μm microfluidic device semi-coated with “ST_A_10” [100 pmol] (here, this is a biotin aptamer which is present with 100 μmol in the stationary phase) with respect to Jurkat cells in the mobile phase (PBS buffer).

TABLE

| 4 times | | | |
|---|---|---|---|
| objective lens | 100 pmol ST_A_10 | | |
| without coating | 800 × 20 μm | with coating | |
| measurement | Time [VCL] | measurement | Time [VCL] |
| 1. | 388 | 1. | 221 |
| 2. | 327 | 2. | 214 |
| 3. | 321 | 3. | 211 |
| 4. | 347 | 4. | 216 |
| 5. | 523 | 5. | 210 |
| 6. | 401 | 6. | 205 |
| 7. | 329 | 7. | 197 |
| 8. | 439 | 8. | 138 |
| 9. | 369 | 9. | 206 |
| 10. | 480 | 10. | 169 |
| 11. | 299 | 11. | 200 |
| 12. | 309 | 12. | 224 |
| 13. | 408 | 13. | 203 |
| 14. | 462 | 14. | 245 |
| 15. | 316 | 15. | 219 |
| average | 381.2 | average | 205.2 |

Time in s/Retention time
Left: window 1, Right: window 2

### DESCRIPTION OF THE FIGURES

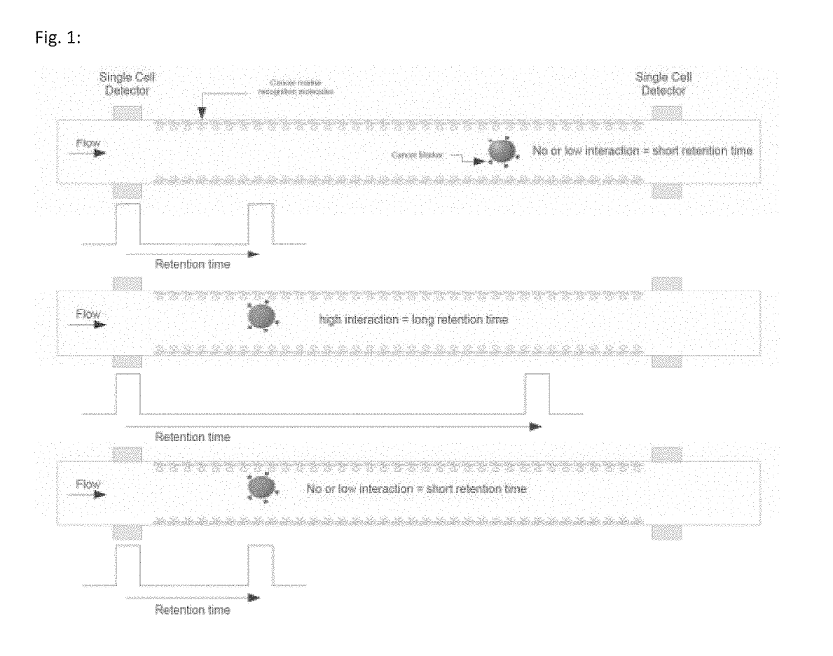

FIG. 1 describes the principle of the invention.

FIG. 2 describes a microfluidic device having a number of areas of different receptors for tumour markers for characterisation of cancer cells, or diagnosis of cancer.

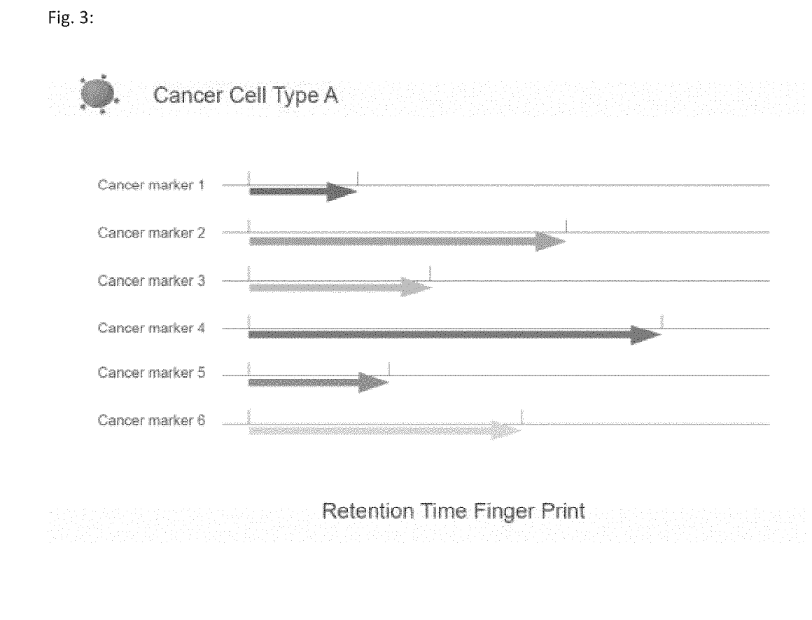

FIG. 3 shows a profile or fingerprint according to the invention.

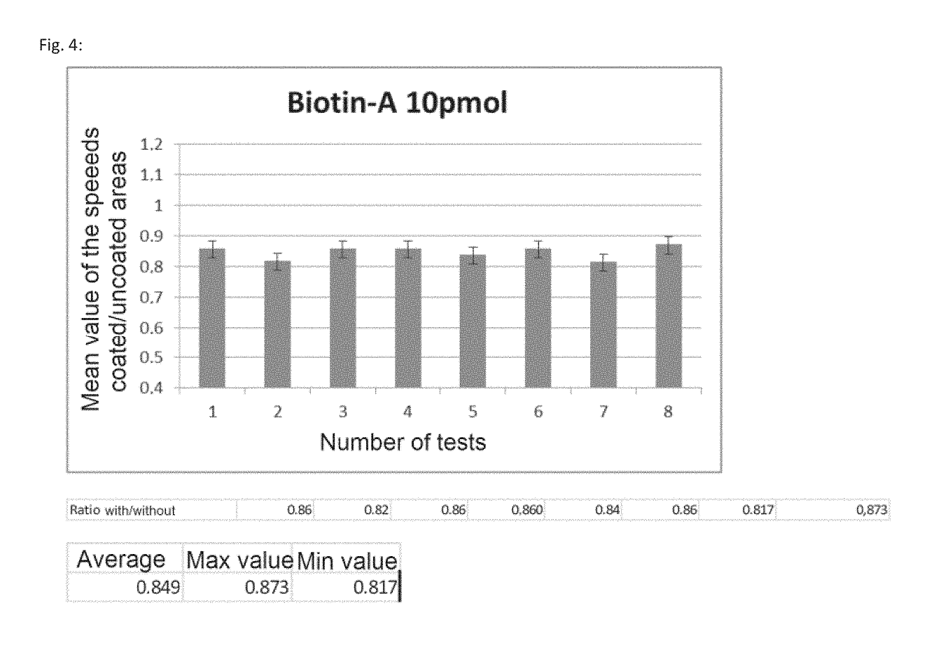

FIG. 4 shows the reproducibility of the retention times or speeds of the biotin aptamer ST A 10 with respect to Jurkat cells on the basis of a microfluidic device according to Example 1.

FIG. 5 shows the dependency of the speed ratio (coating/without coating) of the biotin aptamer with respect to Jurkat cells. Without coating, the ratio is 1, with 10 μmol coating it is 0.85, with 50 μmol coating it is 0.65, with 100 pmol coating it is 0.54, approaching a straight line (=asymptote).

1-14. (canceled)

15. A method for the diagnosis or risk stratification of cancer, wherein the retention times of cancer cells are determined and a profiling of cancer cells is performed.

16. The method of claim 15, wherein a characterization of cancer cells is performed.

17. The method of claim 15, wherein the cancer cells are CTCs.

18. The method of claim 15, wherein the retention time is determined by a microfluidic device or a micro channel.

19. The method of claim 15, wherein the average retention times of cancer cells are determined.

20. The method of claim 18, wherein the microfluidic device or the micro channel has at least one receptor for a tumour marker, in particular polyclonal or monoclonal antibodies, aptamers, peptides, or proteins.

21. The method of claim 18, wherein the microfluidic device or the micro channel has a number of areas of different receptors for tumour markers.

22. The method of claim 15, wherein the retention time is determined by means of cell detection, in particular by means of optical cell detection, for example using a microscope or a fluorescence microscope.

23. The method of claim 15, wherein the retention time is determined by means of a cell detection, in particular by means of computer-assisted readout, more specifically a microscope combined with a camera, with use of cell detection software.

24. The method of claim 15, wherein a profiling of individual cancer cells is performed so as to obtain a profile or fingerprint.

25. The method of claim 15, wherein individual profiles are stored in a database and are compared with one another.

26. The method of claim 15, for the diagnosis and/or risk stratification and identification of patients with increased risk and/or an unfavourable prognosis of cancerous conditions, in particular in the case of symptomatic and/or asymptomatic patients, in order to make clinical decisions, such as further treatment and therapy by means of drugs, in particular in time-critical intensive care or emergency care, including the decision to hospitalise the patient.

27. The method of claim 15, for the prognosis, differential diagnostic early detection and detection, assessment of the degree of severity, and assessment alongside therapy of the progression of a cancerous condition.

28. The method of claim 15, wherein, in a first step, cancer cells are enriched from a body sample and the retention times are then determined.

29. A kit comprising a microfluidic device for determining retention times of cancer cells for carrying out the method of claim 15.

30. The kit of claim 29, comprising means for computer-assisted readout of the microfluidic device, in particular a microscope combined with a camera, and cell detection software.
